(19) United States
(12) Patent Application Publication
(10) Pub. No.: US 2009/0084387 A1
(43) Pub. Date: Apr. 2, 2009

(54) PIECE OF FURNITURE FOR SEXUAL INTERCOURSE

(75) Inventors: Jaume Casteras Farre, Anglesola (ES); Josep M. Petit Agusti, Tarrega (ES)

Correspondence Address: BROWN & MICHAELS, PC;400 M & T BANK BUILDING, 118 NORTH TIOGA ST, ITHACA, NY, 14850, US

(73) Assignee: STONES & STICKS, S.L., Anglesola (ES)

(21) Appl. No.: 12/238,582

(22) Filed: Sep. 26, 2008

(30) Foreign Application Priority Data
Sep. 27, 2007 (ES) U200701959
Jun. 26, 2008 (ES) U200801366

(51) Int. Cl. A61G 15/00; A61F 5/00

(52) U.S. Cl. 128/845; 600/38

(57) ABSTRACT

The invention relates to a piece of furniture for sexual intercourse. The piece of furniture comprises a lower base (1) with two pairs of considerably parallel legs (4), a pair of side columns (2a, 2b) being located on said base (1), which columns respectively have, at their upper part, a T-shaped stringer (36), the parallel stringers (36) or the columns (2a, 2b) being connected to one another by at least one crosspiece profile (65); and in that the legs (4), base (1), columns (2a, 2b), stringers (36) and/or crosspiece profile (63) have a plurality of holes and anchoring points for fixing ropes, chains, harnesses and accessories (5) in general.

### OBJECT OF THE INVENTION

The present invention relates to a piece of furniture for sexual intercourse with a great functionality and polyvalence with a robust construction.

### BACKGROUND OF THE INVENTION

It is currently known that many couples use different apparatuses during sexual intercourse for their mutual enjoyment. This intercourse, in which different types of objects are used, includes sadism, in which one spouse can tie or secure his or her partner for several purposes by means of chains, ropes or other accessories.

Horses, rings and other instruments are normally used for this practice, which are very bulky, must be anchored to the wall or to very eye-catching supports and the generally crude appearance of which gives away their use, not being very discreet.

Different types of “love furniture” are also known which are formed by reclining armchairs with different configurations but still have the problem that when they are not being used, they occupy a very large space, in addition to the fact that their strange appearance does not make them attractive for their incorporation into the decor of the house.

### DESCRIPTION OF THE INVENTION

The piece of furniture for sexual intercourse, object of this invention, has technical particularities intended to offer a wide variety of possibilities of use during sexual intercourse.

The piece of furniture mainly comprises a lower base with two pairs of considerably parallel legs, a pair of side columns being located on said base, which columns respectively have, at their upper part, a T-shaped stringer, the parallel stringers or the columns being connected to one another by at least one crosspiece profile. This structure is thus resistant and allows defining lower, side and upper fixing points both towards the front part and towards the rear part, which are suitable for placing chains, ropes and accessories which can be used during sexual intercourse.

The legs, base, columns, stringers and/or crosspiece profile thus have a plurality of anchoring holes for fixing ropes, chains, harnesses and accessories in general, whereby the accessibility of the piece of furniture in general increases. With this configuration, the piece of furniture allows placing a person in multiple body positions, and even suspended in mid-air.

In a first embodiment, the piece of furniture is designed to show a moderate size, a discreet appearance and to make it go unnoticed when it is not in use, allowing storing accessories and removable elements therein. This piece of furniture comprises the base configured with a chest, with a lid for accessing its inside, respective upward columns being located at two side ends of the chest, between which columns there are, in a detachable and positionable manner, intermediate elements anchored at anchoring points and holes present in said columns.

In the use arrangement of the piece of furniture, additional T-shaped supports configuring the upper stringers are coupled on the columns, these additional supports being supported by means of couplings and front and rear ties. The chest has at the lower part suitable coupling means for removably coupling the front and rear legs, allowing the arrangement of the multiple accessories in a plurality of anchoring points existing on the legs. The piece of furniture is suitable for disassembling said removable elements and storing them in the chest, reducing its size and offering discreet aesthetics when it is not in use.

The piece of furniture can in turn be easily disassembled and the removable parts can be stored inside the chest, reducing the space occupied and having a discreet appearance which does not give away its use. Thus, when not in use, only the lower chest with the upward projecting pillars and the intermediate elements arranged like the backrest of a rustic wood seat, for example, is observed.

It has been provided that the intermediate elements comprise the two halves of a planar clamp, respectively formed by a rigid plate, made of wood for example, having recesses in its edge. The two halves opposed by the edges of the recesses thus allow immobilizing a person by his or her limbs or by the neck while using the piece of furniture for the mentioned purposes, but with the opposite arrangement thereof, it configures part of the supposed backrest or adornment thereof in a discreet manner when it is not used.

Likewise, the piece of furniture can also comprise at least one padded crosspiece between the intermediate elements, which allows placing a person lying on his or her stomach on top of the piece of furniture or being used as part of the backrest when the piece of furniture is not used.

The piece of furniture also comprises a reinforcing crosspiece profile, having at its ends anchorings for its arrangement between the lower pillars of the piece of furniture or at the upper part between the two additional supports, acting as a reinforcing bridge to the structure once it is assembled.

The extendable legs allow providing the piece of furniture with the sufficient stability when it is extended as much as possible and is subjected to the stress of its use. These legs are coupled by removable coupling means, such as slots open at at least one front face, whereby two front legs and two rear legs are used. These legs have in their entire length the mentioned anchoring points, for example holes, for fixing and tightening the accessories, such as chains, ropes, pulleys, snap hooks and other elements.

A reinforcing stringer can be found between the aforementioned slots, which stringer has a plurality of holes for assembling bars with an anchoring eye at their projecting end, thus allowing to provide the lower part of the chest with more anchoring and fixing points.

In a preferred embodiment, the pillars are formed by two parallel planar elements in at least part of their length, for example thick boards, having a plurality of through holes configuring the anchoring points for anchoring the intermediate elements and other accessories in a simple manner by means of pins.

Likewise, each additional support is formed by a post having at its upper part an open slot passing from its front face to its rear face, in which the stringer or element transverse thereto is housed. Once this stringer is arranged on the post, it is locked by means of the ties arranged from the proximity of its ends to the lower column. The stringer also has therealong the holes forming the aforementioned anchoring points, from which the accessories, chains, ropes and others can be hung.

The coupling between the additional support and the lower column is formed by a side flat bar coupled to the lower end or post of the additional support, this flat bar being extended in a lower projection housed inside the column. The projection of the side flat bar has at least one through hole of a securing pin of a tie at its lower end aiding in the stability and firmness of the assembly of the additional support on the column.

The column can have at its upper end an angular, arrow-shaped or rounded shape contributing to its aesthetic appearance, while the base of the post of the additional support also has a complementary groove in the area of the coupling flat bar, forming a more solid attachment fitting.

In a second embodiment of the piece of furniture, the lightness and the manufacturing simplicity has been emphasized, allowing the configuration of a non-removable fixed structure, made mainly in high-strength metal profiles, more suitable for its more or less fixed installation in a specific room for its use.

The piece of furniture for sexual intercourse thus comprises the base configured by the pair of longitudinal legs respectively attached to tubular slots arranged at each of the ends of a crosspiece element, the pair of side columns respectively having the T-shaped stringer projection being located on said base. The stringers are connected to one another in a parallel manner by structurally stiffening crosspiece profiles.

Likewise, the legs, columns, stringers, crosspiece profiles and/or crosspiece elements have the holes for fixing ropes, chains, harnesses and other accessories. Thus, the piece of furniture likewise allows tying a person in multiple positions and even suspending him or her in mid-air with complete safety, without needing to use anchorings fixed to walls and ceilings, but rather the piece of furniture configures the self-supporting structure with the suitable strength.

All the elements are formed by high-strength metal profiles, which allow a robust use once the piece of furniture is assembled.

Each leg is thus formed by two halves coupled by each end of the slot by means of screws passing through holes for that purpose. Once these legs are made integral with the crosspiece element, they define a firm support surface and their height is reduced to allow the movement of persons around them without much inconvenience.

For example, in one embodiment of the invention, each column is formed by an outwardly oriented omega-shaped profile having a U-shaped profile oriented in the opposite direction associated at its back. This constitution allows providing the column with an optimized resistant section to prevent its bending when it is loaded with the weight of a person, said column being lighter than a solid wood column. Furthermore, both the U-shaped profile and the outer flanges of the omega-shaped profile have a large amount of holes acting as coupling points for coupling the ropes and securing accessories which can be used in sexual intercourse.

Each column has at its lower end a planar plate for fixing on the slot of the base. This plate has holes for placing screws for the purpose of ensuring said fixing. A support of the upper part of the piece of furniture which is simple to carry out and effective is thus obtained.

In an embodiment, the column is formed by two halves joined by means of a side plate, whereby the disassembled transport of the piece of furniture to the installation site and the handling of a column which can have a considerable height is facilitated.

Each upper stringer is formed by a rectangular profile having a planar bracket associated thereto at the side part. This side profile has a sufficient bending strength to support the load which can occur upon leaving a person suspended in mid-air by means of chains or ropes, and the bracket allows having the suitable anchoring holes, without said profile being weakened.

The bracket has a variable section, with a greater width at the central part than at the ends, the holes for fixing accessories in the stringers being located at the lower part of said bracket.

The crosspiece profiles are fixed at the ends of the stringers, stabilizing the structure of the piece of furniture at the upper part. Each of said crosspiece profiles is formed by a U-shaped profile, in the flanges of which the holes for fixing the accessories are preferably located, allowing new anchoring points for anchoring ropes and chains or accessories.

### DESCRIPTION OF THE DRAWINGS

To complement the description which is being made and with the aim of aiding to better understand the features of the invention, a set of drawings is attached to this specification in which the following has been shown with an illustrative and non-limiting character:

FIG. 1 shows an elevational view of the convertible piece of furniture in a folded position, having the discreet appearance of a chest with a rear backrest.

FIG. 2 shows a section profile view of the piece of furniture of the previous figure.

FIG. 3 shows a profile view of the piece of furniture of the previous figures.

FIG. 4 shows a perspective view of the convertible piece of furniture extended in a use arrangement with a slight representation of accessories fixed at several points of its structure.

FIG. 5 shows an elevational view of the piece of furniture of the previous figure and a detail of the assembly of the legs and the bars which can be coupled on the chest.

FIG. 6 shows a detail of fixing the additional support and the ties on a pillar.

FIG. 7 shows an elevational view of the reinforcing crosspiece and its anchorings.

FIG. 8 shows an elevational view of the two opposing halves of the planar clamp and their anchorings.

FIG. 9 shows an elevational view of the padded crosspiece and its anchorings.

FIG. 10 shows an elevational view of the piece of furniture in its fixed configuration.

FIG. 11 shows a plan view of the piece of furniture of the previous view.

FIG. 12 shows a profile view of the piece of furniture of FIG. 10.

FIG. 13 shows a perspective view of the piece of furniture of FIG. 10.

### PREFERRED EMBODIMENT OF THE INVENTION

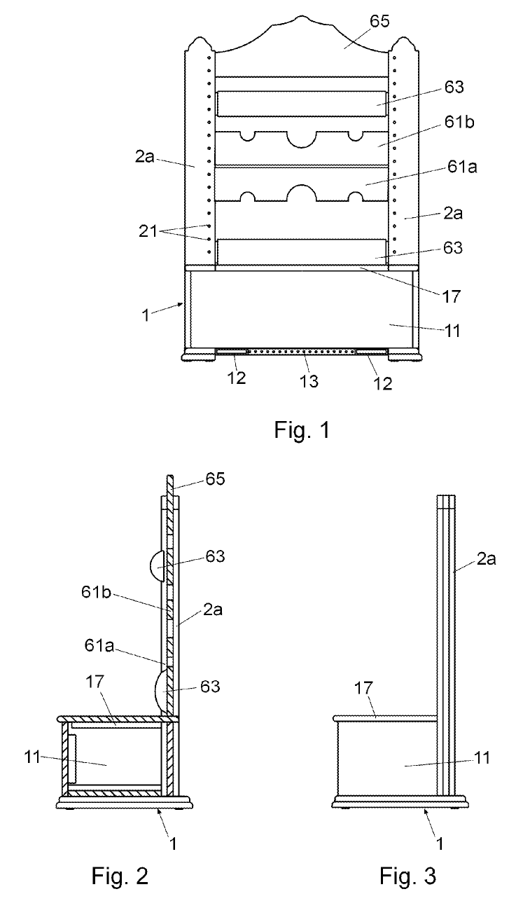

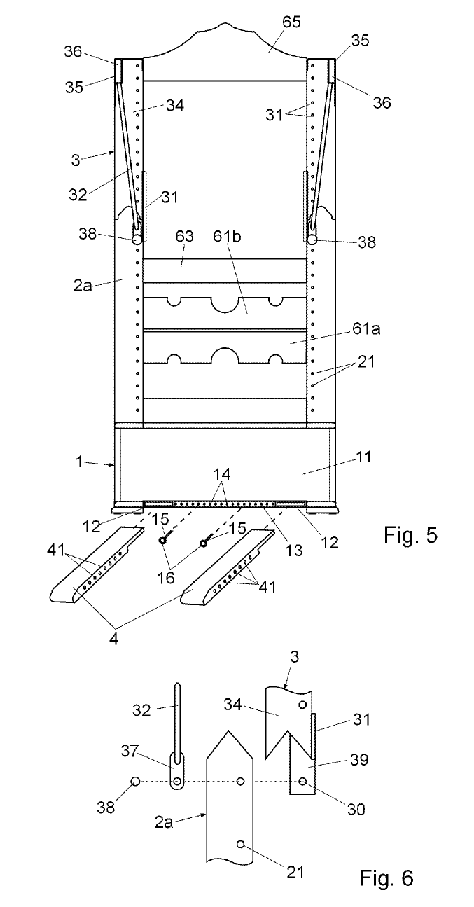

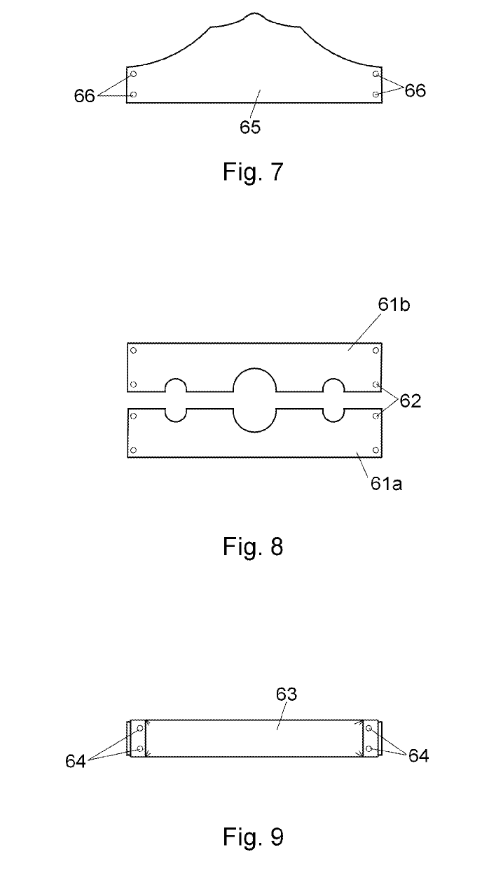

As can be observed in the referenced figures, the piece of furniture made according to a first embodiment extendable from a reduced and decorative configuration when it is not in use to an extended configuration for its direct use and the coupling of the suitable accessories, thus depicted in FIGS. 1 to 9, comprises a lower base (1) formed by an elongated chest (11), in this case made of wood, having at two side ends two upward columns (2a), preferably in a parallel manner, on which there are arranged additional supports (3) with a T-shaped constitution by means of respective couplings (31) which are removable and are secured by means of ties (32) at an angle. The base (1) has at the lower part of the chest (11) front and rear legs (4) extended in a considerably horizontal manner, for example made of stainless steel, from removable coupling means providing the piece of furniture with the necessary stability during its extended use.

In the columns (2a), additional supports (3) and legs (4), there are a plurality of anchoring points (21, 33, 41) defined as holes in which the accessories (5) which can be used with the piece of furniture, such as chains, ropes, fixing snap hooks, pulleys and others, can be coupled.

The columns (2a) are respectively formed by two parallel planar elements, in this case made of wood, on which there are holes arranged as opposing through holes, which holes configure a vertical line of anchoring points (21), the upper end of said columns (2a) being able to be finished with a decorative angled or rounded shape for coupling with a conjugate shape at the lower end of the contiguous additional support (3).

Intermediate elements can be placed between the columns (2a) at different height and in a different manner, which elements comprise a planar clamp formed by two halves (61a, 61b) configured by rigid plates having in a longitudinal edge rounded entrapment recesses when said halves are opposed. Both halves (61a, 61b) of the clamp have at their ends holes or anchorings (62) for securing to the holes of the anchoring points (21) of the pillars (2a) by means of pins. Furthermore, said intermediate elements also comprise two padded support crosspieces (63), which also have at their end holes or anchorings (64) to the pillars (2a) by means of pins.

Another additional element is a reinforcing crosspiece profile (65) for its arrangement between the upper ends of the columns (2a) and between the upper ends of the additional supports (3) to improve the stability of the piece of furniture, holes or anchorings (66) to the columns (2a) and to the additional supports (3) being located at the ends of this reinforcing crosspiece profile (65).

Each additional support (3) is formed by a vertical post (34) having at its upper part and open slot (35) passing from its front face to its rear face, in which a stringer (36) is removably housed, which stringer has a termination of an attached tie (32) coupled in proximity with each of its ends, said tie (32) having at the opposite end a plate (37) which can be arranged on the upper end of the pillar (2a) by means of at least one locking pin (38), thus enabling the fixing of the post (34) of the additional support (3) as the two front and rear ties (32) are grouped by means of said pins (38) on a coupling (31) existing at the lower end of the post (34) of the additional support (3) with the lower pillar (2). This coupling (31) is formed by a side flat bar coupled to the shaped lower end of the post (34) of the additional support (3), and extended in an inner projection (39) housed inside the column (2a). In this projection (39) there is at least one through hole (30) of the securing pin (38) of the plate (37) of the ties (32) that are close, whereby the greatest possible integrality is achieved.

At the lower part of the base (1) and the chest (11), there are two slots (12) at each larger side, forming the temporary coupling means for coupling the extended legs (4). These slots (12) are open at their front face, there being between them a reinforcing metal stringer (13) having a plurality of holes (14) for housing bars (15) with an eye like an anchoring point (16) at their end.

Each leg (4), in this case made of stainless steel, has along its entire length a series of holes forming the anchoring points (21) for anchoring the accessories (5) which are to be used.

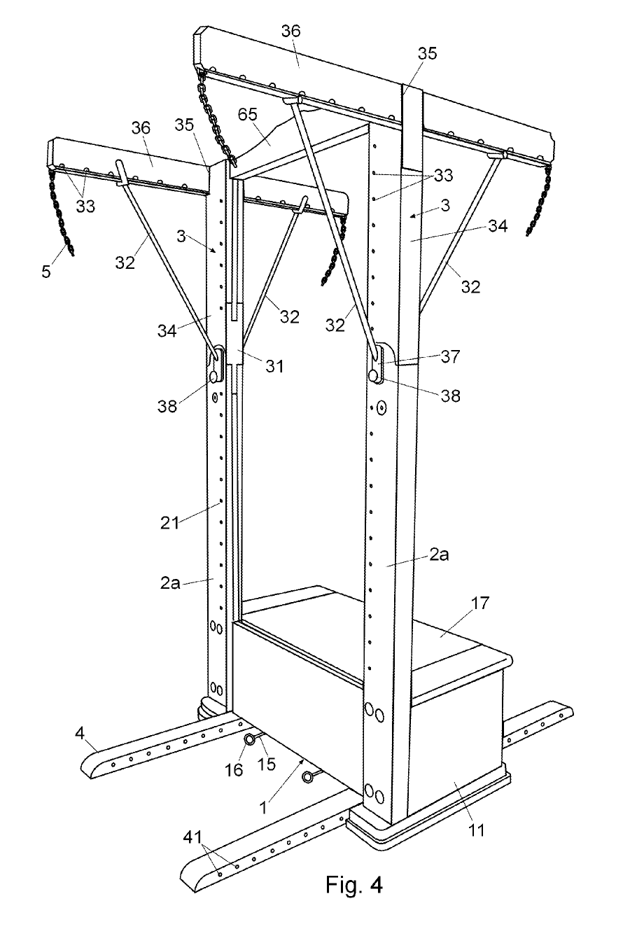

FIGS. 1 and 3 show how the convertible piece of furniture is arranged when it is not in use, with the additional supports (3), the legs (4) and the accessories (5) stored inside the chest (11), which can be accessed by an upper lid (17), and the two padded crosspieces (63) and the two halves (61a, 61b) of the clamp, placed in an opposite arrangement to configure a decorative backrest, being assembled on the pillars (2a).

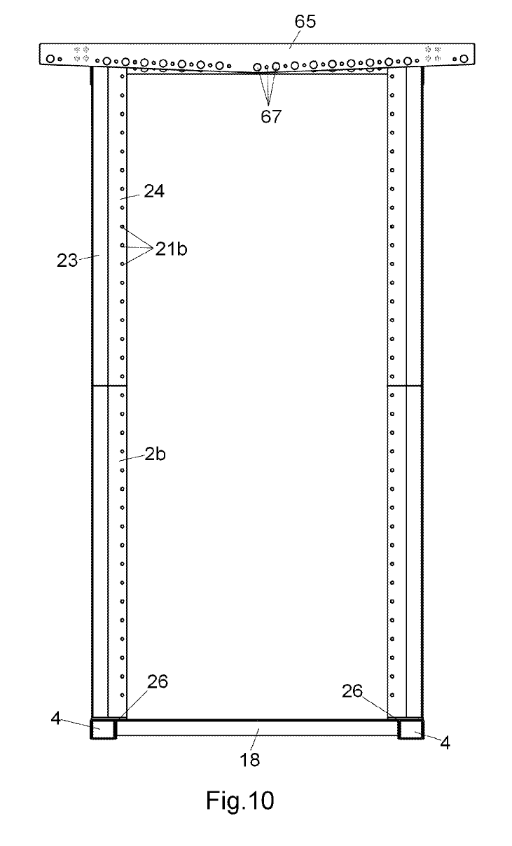

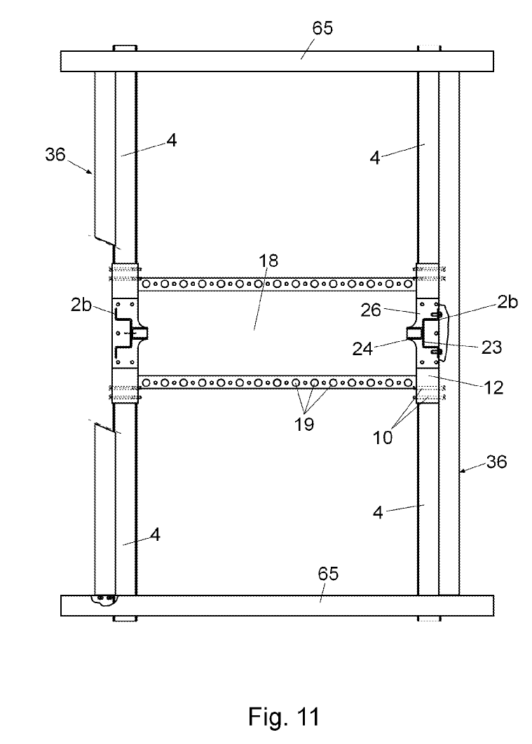

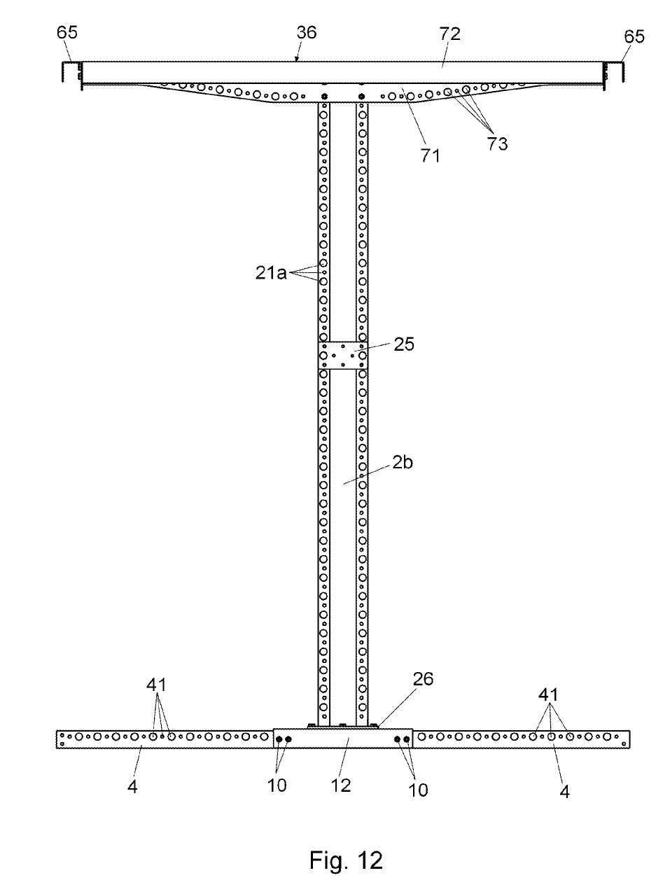

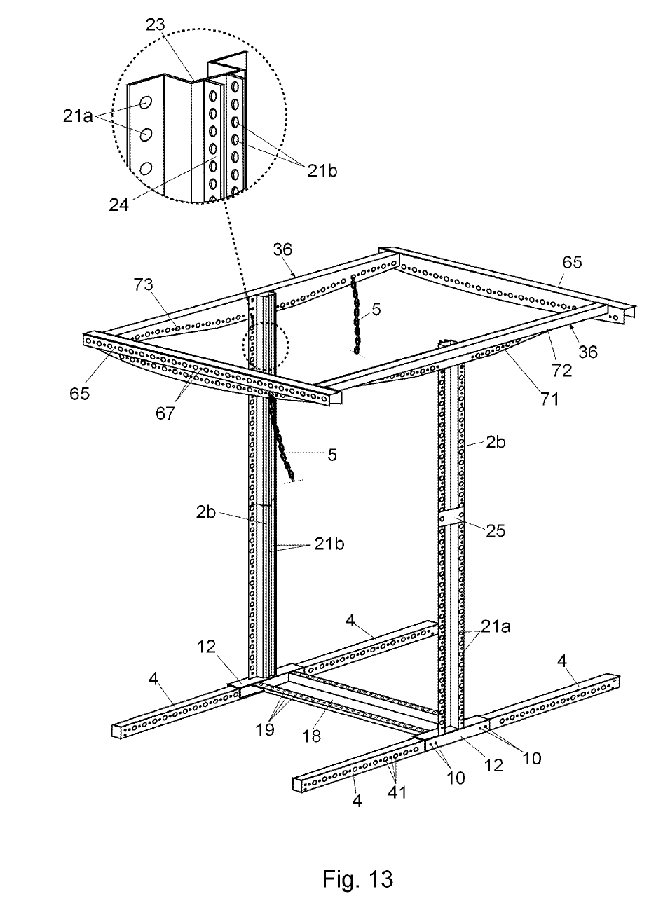

In a second embodiment with a fixed structure and simpler constitution based on metal profiles, thus shown in FIGS. 10 to 13, the piece of furniture is configured by the base (1) with longitudinal legs (4) and a crosspiece element (18), columns (2b) arranged on said base (1) and which at its upper part comprises two stringers (36) arranged in a T-shape and crosspiece profiles (65) between the ends of the stringers (36), such that the structure is stiffened. In said legs (4), crosspiece element (18), columns (2b), stringers (36) and crosspiece profiles (65) there is also defined a plurality of holes and anchoring points (19, 21a, 21b, 41, 67, 73) for fixing and anchoring accessories (5) such as chains, ropes, pulleys and others which allow immobilizing and even suspending a person in mid-air during sexual intercourse.

The legs (4) can be formed in one piece or, as shown in the attached embodiment, by two halves attached at the ends opposing a U-shaped coupling slot (12) existing on each side of the lower crosspiece element (18), said lower crosspiece element (18) being configured by an intermediate profile with an omega-shaped section, which is considerably wide and has a small height, with the holes (19) distributed along both longitudinal flanges. Said attachment of each half of the legs (4) with the slot (12) is carried out by means of transverse through screws (10).

Each column (2b) is formed by an outwardly oriented omega-shaped profile (23) with a certain width, a profile (24) with a U-shaped profile being attached at its back corresponding to the inner part of the piece of furniture. Both the flanges of the outer omega-shaped profile (23) and the flanges of the inner profile (24) have respective lines of anchoring holes (21a, 21b) for anchoring accessories (5). In the example shown, the column (2b) is formed by two halves joined at mid-height by means of a side plate (25), while the lower half has at its free end a planar plate (26) for fixing on the slot (12) of the crosspiece element (18).

Each upper stringer (36) is formed by a longitudinal bracket (71) and an adjacent rectangular profile (72). The bracket (71) is fixed to the outer face of the upper part of an adjacent column (2b), more specifically to the flanges of the profile (23), the rectangular profile (72) being located on the opposite face of said bracket (71). The bracket (71) has a variable section, being wider at its central part and narrowing towards the ends, the anchoring holes (73) being made next to the lower edge or rim of said bracket (71), on both side of the column (2b).

The crosspiece profiles (65) are fixed at the ends of the rectangular profiles (72) of the stringers (36). These crosspiece profiles (65) have a U-shaped section configuration, with a longer flange, and in which the holes (67) for fixing the accessories (5) are made.

Having sufficiently described the nature of the invention, as well as a preferred embodiment, it is stated for the relevant purposes that the materials, shape, size and arrangement of the described elements can be modified, provided that this does not involve an alteration of the essential features of the invention which are claimed below.

1. A piece of furniture for sexual intercourse, characterized in that it comprises a lower base (1) with two pairs of considerably parallel legs (4), a pair of side columns (2a, 2b) being located on said base (1), which columns respectively have, at their upper part, a T-shaped stringer (36), the parallel stringers (36) or the columns (2a, 2b) being connected to one another by at least one crosspiece profile (65); and in that the legs (4), base (1), columns (2a, 2b) , stringers (36) and/or crosspiece profile (63) have a plurality of holes and anchoring points for fixing ropes, chains, harnesses and accessories (5) in general.

2. The piece of furniture according to claim 1, characterized in that the base (1) comprises a chest (11), with a lid (17) for accessing its inside, respective upward columns (2a) being located at two side ends of the chest (11), between which columns there are, in a detachable and positionable manner, intermediate elements anchored at anchoring points (21) present in said columns (2a), additional T-shaped supports (3) configuring the upper stringers being coupled on the columns (2a) in the use arrangement of the piece of furniture, these additional supports (3) being supported by means of couplings (31) and front and rear ties (32), the chest (11) having at the lower part suitable coupling means for removably coupling the front and rear legs (4), allowing the arrangement of the multiple accessories (5) in a plurality of anchoring points (41) existing on the legs (4), the piece of furniture being operatively suitable for disassembling said removable elements and storing them in the chest (11), reducing its size and offering discreet aesthetics when it is not in use.

3. The piece of furniture according to any of claims 1 and 2, characterized in that the intermediate elements comprises the two halves (61a, 61b) of a planar clamp, formed by a rigid plate having recesses in its longitudinal edge.

4. The piece of furniture according to any of claims 1 and 2, characterized in that the intermediate elements comprise at least one padded crosspiece (63).

5. The piece of furniture according to claim 2, characterized in that the crosspiece profile (65) has at its ends anchorings (66) for its arrangement between the lower columns (2a) or at the upper part between the additional supports (3).

6. The piece of furniture according to claim 1, characterized in that the coupling means for coupling the legs (4) are slots (12) open at at least one front face.

7. The piece of furniture according to claim 6, characterized in that between the slots (12) of the coupling means there is a stringer (13) with a plurality of holes (14) for housing bars (15) with an eye configuring an anchoring point (16) at their projecting end.

8. The piece of furniture according to claim 2, characterized in that the columns (2a) are formed in at least part of their length by two parallel planar elements having a plurality of through holes, configuring the anchoring points (21) for anchoring the intermediate elements.

9. The piece of furniture according to claim 2, characterized in that each additional support (3) is formed by a post (34) having at its upper part an open slot (35) passing from its front face to its rear face, in which a transverse element or stringer (36) is housed.

10. The piece of furniture according to any of claims 2, 8 and 9, characterized in that the coupling (31) between the additional support (3) and the lower column (2a) is formed by a side flat bar coupled to the lower end or post (34) of the additional support (3), and extended in an inner projection (39) which can be housed inside the column (2a).

11. The piece of furniture according to claim 10, characterized in that the projection (39) has at least one through hole (30) of a securing pin (38) of a tie (32) at its lower end.

12. The piece of furniture according to claim 1 characterized in that the base (1) is configured by the pair of longitudinal legs (4) respectively attached to tubular slots (12) arranged at each of the ends of a crosspiece element (18), the pair of side columns (2b) respectively having at their upper part the projecting T-shaped stringer (36) being located on said base (1), the parallel stringers (36) being connected to one another by structurally stiffening crosspiece profiles (65).

13. The piece of furniture according to claim 12, characterized in that each leg (4) is formed by two halves coupled at each end of the slot (12) by means of screws (10) passing through holes for that purpose.

14. The piece of furniture according to claim 12, characterized in that each column (2b) is formed by an outwardly oriented profile (23) with an omega-shaped section with a certain width, having a U-shaped profile (24) in the opposite direction associated at its back.

15. The piece of furniture according to claim 12, characterized in that each column (2b) has at its lower end a planar plate (26) for fixing on the slot (12) of the base.

16. The piece of furniture according to claim 12, characterized in that the column (2b) is formed by two halves joined by means of a side plate (25).

17. The piece of furniture according to claim 12, characterized in that each upper stringer (36) is formed by a rectangular profile (72) having a planar bracket (71) associated at the side part.

18. The piece of furniture according to claim 17, characterized in that the bracket (71) has a variable section, with a greater width at the central part than at the ends, the holes (73) for fixing accessories (5) in the stringers (36) being located in said bracket (71).

19. The piece of furniture according to claim 12, characterized in that the crosspiece profiles (65) are fixed at the ends of the stringers (36).

20. The piece of furniture according to claim 12, characterized in that each of the crosspiece profiles (65) is formed by a profile with a U-shaped section having in its flanges the holes (67) for fixing the accessories (5).
